(19) United States
(12) Patent Application Publication
(10) Pub. No.: US 2025/0191768 A1
(43) Pub. Date: Jun. 12, 2025

(54) COMBINATION OF BIOMARKERS, AND METHOD FOR DETECTING COGNITIVE DYSFUNCTION OR RISK THEREOF BY USING SAID COMBINATION

(71) Applicant: MCBI INC., Ibaraki (JP)

(72) Inventors: Hitomi ITO, Ibaraki (JP); Shan LIU, Ibaraki (JP)

(73) Assignee: MCBI INC., Ibaraki (JP)

(21) Appl. No.: 18/036,034

(22) PCT Filed: Nov. 10, 2021
(86) PCT No.: PCT/JP2021/041332

§ 371 Date: Nov. 3, 2023

Related U.S. Application Data
(60) Provisional application No. 63/111,796, filed on Nov. 10, 2020.

(51) Int. Cl. G16H 50/30; G01N 33/68; G16H 50/20

(52) U.S. Cl. CPC G16H 50/30; G01N 33/6896; G16H 50/20; G01N 2333/4709; G01N 2333/4716; G01N 2333/775; G01N 2800/2814; G01N 2800/50; G01N 2800/56

(57) ABSTRACT

[Problems] The purpose of the present technology is to accurately detect cognitive impairment or risk thereof.

[Solution] The present technology provides a cognitive impairment determination system comprising an information processing device that executes a determination step of determining presence or absence cognitive impairment or risk of cognitive impairment, based on amounts of the following biomarkers (a), (b), (c), (d), and (e) contained in a biological sample: (a) a biomarker consisting of an intact protein of Apolipoprotein A1 comprising an amino acid sequence represented by SEQ ID NO: 1, or a partial peptide thereof, (b) a biomarker consisting of an intact protein of Transthyretin comprising an amino acid sequence represented by SEQ ID NO: 2, or a partial peptide thereof, (c) a biomarker consisting of an intact protein of Complement C3 having an amino acid sequence represented by SEQ ID NO: 3, or a partial peptide thereof, (d) a biomarker Aβ1-40 consisting of a peptide having an amino acid sequence represented by SEQ ID NO: 4, and (e) a biomarker Aβ1-42 consisting of a peptide having an amino acid sequence represented by SEQ ID NO: 5.

### TECHNICAL FIELD

The present technology relates to a combination of biomarkers, in particular a combination of biomarkers suitable for detecting cognitive impairment or risk thereof. The present technology also relates to a method for detecting cognitive impairment or risk thereof using the combination.

### BACKGROUND ART

As a means for discriminating a difference between samples exhibiting normal or non-normal condition of the living organisms, the main conventional technology is a technique that has been generally used for in vitro diagnostic agents. Most of the in-vitro diagnostic agents are used for diagnostic tests by analyzing components in blood as biomarkers. In the prior art in this field, an amount of a single specific protein or a so-called oligopeptide with a molecular weight of 10,000 or less in blood is measured or, in a case of an enzyme protein, its activity is measured, and its diagnosis has been aided by an apparent difference between normal (healthy) samples and diseased samples. That is, an amount of a single or a plurality of specific proteins or specific oligopeptides or an amount of activity thereof in a certain number of biological samples derived from healthy and diseased patients are measured in advance, and a range of abnormal value and a range of normal value are decided. Next, a biological sample to be evaluated is measured by the same method, and examination evaluation is performed depending on whether the measurement result belongs to any of the decided range of abnormal value and the decided range of normal value.

Regarding biomarkers used for detecting cognitive impairment, for example, Patent Literature 1 below discloses (a) a biomarker for detecting a cognitive impairment disease including an intact protein of Apolipoprotein A1 containing an amino acid sequence represented by SEQ ID NO: 1, or a partial peptide thereof, (b) a biomarker for detecting a cognitive impairment disease including an intact protein of Transthyretin containing an amino acid sequence represented by SEQ ID NO: 2, or a partial peptide thereof, and (c) a biomarker for detecting a cognitive impairment disease including an intact protein of Complement C3 containing an amino acid sequence represented by SEQ ID NO: 3, or a partial peptide thereof.

### CITATION LIST

### Patent Literature

Patent Literature 1: International Publication No. 2014-207888

### SUMMARY OF INVENTION

### Technical Problem

An object of the present technology is to accurately detect cognitive impairment or risk thereof.

### Solution to Problem

The present inventors have found that specific biomarker combinations are suitable for detecting cognitive impairment or risk of cognitive impairment.

That is, the present technology provides a combination of the following biomarkers (a), (b), (c), (d), and (e):
(a) a biomarker consisting of an intact protein of Apolipoprotein A1 comprising an amino acid sequence represented by SEQ ID NO: 1, or a partial peptide thereof;
(b) a biomarker consisting of an intact protein of Transthyretin comprising an amino acid sequence represented by SEQ ID NO: 2, or a partial peptide thereof;
(c) a biomarker consisting of an intact protein of Complement C3 having an amino acid sequence represented by SEQ ID NO: 3, or a partial peptide thereof;
(d) a biomarker Aβ1-40 consisting of a peptide having an amino acid sequence represented by SEQ ID NO: 4; and
(e) a biomarker Aβ1-42 consisting of a peptide having an amino acid sequence represented by SEQ ID NO: 5.

The combination may be used for detection, diagnosis or determination of cognitive impairment or risk thereof. The combination may be used for detection, diagnosis, or determination of cognitive decline.

The present technology also provides a combination of the following biomarkers (a), (b), (c), (d), (e), and (f):
(a) a biomarker consisting of an intact protein of Apolipoprotein A1 comprising an amino acid sequence represented by SEQ ID NO: 1, or a partial peptide thereof;
(b) a biomarker consisting of an intact protein of Transthyretin comprising an amino acid sequence represented by SEQ ID NO: 2, or a partial peptide thereof;
(c) a biomarker consisting of an intact protein of Complement C3 comprising an amino acid sequence represented by SEQ ID NO: 3, or a partial peptide thereof;
(d) a biomarker Aβ1-40 consisting of a peptide having an amino acid sequence represented by SEQ ID NO: 4;
(e) a biomarker Aβ1-42 consisting of a peptide having an amino acid sequence represented by SEQ ID NO: 5; and
(f) a biomarker consisting of an intact protein of BACE1 comprising an amino acid sequence represented by SEQ ID NO: 6, or a partial peptide thereof.

The combination may be used for the detection, diagnosis or determination of cognitive impairment or risk thereof. The combination may also be used for detection, diagnosis, or determination of cognitive decline.

The present technology also provides a method of detecting, diagnosing, or determining cognitive impairment or risk thereof.

The present technology also provides a method of detecting, diagnosing, or determining cognitive decline.

The present technology also provides a method of determining a degree of progression of cognitive impairment.

These methods may comprise a step of detecting, diagnosing, or determining cognitive impairment or risk thereof based on amounts of biomarkers constituting the above-mentioned combination in a human biological sample.

In these methods, preferably, amounts of biomarkers (a), (b) and (c) and a ratio Aβ40/Aβ42 of amounts of biomarkers (d) and (e) are used. More preferably, in the present method, amounts of biomarkers (a), (b) and (c), the ratio Aβ40/Aβ42 of amounts of biomarkers (d) and (e), and an amount of a biomarker (f) are used.

The present technology also provides a method of using the combination of biomarkers for the detection of cognitive impairment or risk thereof. The present technology also provides a method of using the combination of biomarkers to detect, diagnose, or determine cognitive decline. The present technology also provides a method of using the combination of biomarkers to determine a degree of progression of cognitive impairment.

In these use methods, preferably, amounts of biomarkers (a), (b) and (c) and a ratio Aβ40/Aβ42 of amounts of biomarkers (d) and (e) are used. More preferably, in the present method, amounts of biomarkers (a), (b) and (c), the ratio Aβ40/Aβ42 of amounts of biomarkers (d) and (e), and an amount of a biomarker (f) are used.

In the method, human cognitive impairment or risk thereof may be detected, cognitive decline may be detected, or a degree of progression of cognitive impairment may be determined, based on these amounts and ratios.

In the method, the cognitive impairment may be mild cognitive impairment or Alzheimer's disease. That is, in the method, the combination may be used for detecting mild cognitive impairment or Alzheimer's disease in a human or for detecting risk that a human suffers from mild cognitive impairment or Alzheimer's disease.

In the method, the cognitive impairment may be mild cognitive impairment or Alzheimer's disease. That is, in the method, the combination mentioned above may be used for detecting whether a human is in any of the following stages: a stage where the human has neither mild cognitive impairment nor Alzheimer's disease, a stage where the human has mild cognitive impairment, and a stage where the human has Alzheimer's disease.

The use methods and the methods of detection, diagnosis or determination may comprise a measuring step of measuring amounts (particularly concentrations) of the biomarkers that constitute the combination in a biological sample. In the measurement, amounts of biomarkers that make up the combination may be measured simultaneously or separately. Preferably, from one biological sample (especially plasma), amounts of biomarkers constituting the combination contained in the biological sample are simultaneously measured. “Simultaneous measurement” may mean measuring amounts of all biomarkers that make up the combination in one measurement procedure (e.g., one ELISA measurement or LC/MS measurement).

The present technology also provides a kit for measuring biomarkers that constitute the combination. The detection kit may comprise an antibody or aptamer against the biomarker.

The present technology also provides a method for deciding a regression model for detecting cognitive impairment or risk thereof, comprising:
a step of acquiring or measuring data on amounts of biomarkers that constitute the combination, contained in each biological sample of a plurality of humans; and
a regression analysis step of performing regression analysis using the presence or absence of cognitive impairment or a stage of cognitive impairment of each of the plurality of humans and the amounts measured for each human and performing fitting to a regression model.

The deciding method may include a detection step of detecting cognitive impairment or risk thereof in a subject using the regression model acquired by the fitting in the regression analysis step.

### Advantageous Effects of Invention

According to the present technology, it is possible to accurately detect or determine cognitive impairment or risk thereof. In addition, according to the present technology, it is possible to detect or determine a degree of cognitive impairment progression in humans.

Note that the effect of the present technology is not necessarily limited to these described in this paragraph, and may be any of effects explained in the present specification.

### BRIEF DESCRIPTION OF DRAWINGS

FIG. 1 is a graph for explaining the relationship between biomarkers and cognitive impairment.

FIG. 2 is a graph for explaining the relationship between biomarkers and cognitive impairment.

FIG. 3 is a graph to illustrate the clinical efficacy of the combination of biomarkers of the present technology.

FIG. 4 is a graph showing the distribution of VSRAD scores and MMSE scores of 363 specimens used in Test Examples 1 and 2.

FIG. 5 is a graph showing the distribution of plasma concentrations of ApoA-1, TTR, and C3 in 363 specimens used in Test Examples 1 and 2.

FIG. 6 is a diagram showing the details of the evaluation result by ROC.

FIG. 7 is a diagram showing the details of the evaluation result by ROC.

FIG. 8 is a diagram showing Context of Use of biomarkers.

FIG. 9 is a diagram showing the action of Sequester protein.

FIG. 10 is a block diagram showing a configuration example of a determination system according to the present technology.

FIG. 11 is a block diagram showing a configuration example of an information processing device according to the present technology.

### DESCRIPTION OF EMBODIMENTS

Hereinafter, embodiments for implementing the present technology will be explained in detail. In addition, the embodiment described below shows an example of a typical embodiment of the present technology, and the present technology is not limited only to these embodiments.

### 1. Description of Related Technology

As a means for discriminating a difference between samples exhibiting normal or non-normal conditions of living organisms, the main conventional technology is a technique that has been generally used for in vitro diagnostic agents.

Most of the in-vitro diagnostic agents are used for conducting a diagnostic examination by analyzing components in blood as biomarkers.

In the prior art in this field, an amount of a single specific protein or so-called oligopeptides with a molecular weight of 10,000 or less in blood has been measured, or in a case of an enzymatic protein, the activity has been measured, and an apparent difference between normal (healthy) and diseased samples has aided diagnosis.

That is, an amount of a single or a plurality of specific proteins or specific oligopeptides or an amount of activity thereof in a certain number of biological samples derived from healthy and diseased patients is measured in advance, and ranges of abnormal values and normal values are decided. Next, the biological sample to be evaluated is measured by the same method, and examination evaluation is performed depending on whether the measurement result belongs to the decided range of abnormal values or normal values.

As a specific measurement method, an Enzyme Linked Immunosorbent Assay (ELISA) in which the sample is used as it is or diluted in advance, and an amount of a single or multiple specific proteins or peptides was measured by an amount of color development of the sample using a specific primary or secondary antibody labeled with an enzyme that develops color when reacted with a substrate; and Chemiluminescent Immunoassay (CLIA); and the like are mentioned. In addition, RADioimmunoassay (RIA) in which an amount of the specific protein or peptide is measured using a radioisotope bound to a primary antibody or secondary antibody; and an enzyme activity measurement method in which if the protein is an enzyme, a substrate is directly added and the product is measured by color development or the like; and the like are mentioned.

There is also a method of analyzing products obtained by enzymatic degradation of substrate, by high performance liquid chromatography (HPLC). There are also LC-MS/MS methods combining HPLC and mass spectrometry, and selected reaction monitoring (SRM)/multiple reaction monitoring (MRM) methods using this method.

In addition, there are also methods in which after subjecting the sample to an appropriate pretreatment, two-dimensional polyacrylamide gel electrophoresis (2D-PAGE) is performed to separate the protein or peptide, and then the target protein or peptide is stained with silver, Coomassie blue, or subjected to immunostaining (Western blotting) using the corresponding antibody, to measure the concentration in the sample.

There is also a method of fractionating a biological sample by column chromatography and analyzing proteins and peptides contained in the fractions by mass spectrometry.

In addition, there are a method of performing mass spectrometry using a protein chip as a pretreatment, instead of column chromatography, and a method of performing mass spectrometry using magnetic beads as a pretreatment.

Furthermore, the present inventor is developing an immunoMS method of binding an antibody against a protein or peptide of interest to beads (including magnetic beads), thereby capturing the protein or peptide to be measured, and then eluting from the beads and measuring by mass spectrometry.

Also, for the purpose of analyzing intact proteins, a method has been reported in which the protein is digested with trypsin or the like and then mass spectrometry is performed by the above method.

However, in either case, using the properties of intact proteins, they are fractionated as they are, or protein molecules that specifically adsorb are selected and analyzed by mass spectrometry.

Cognitive impairment diseases, mainly Alzheimer's disease, are rapidly increasing in Japan as well, along with the recent aging of the population. In 1995, the number was about 1.3 million, but in 2010 it increased to about 2.8 million, and is expected to reach about 4.1 million in 2020. Alzheimer's disease is said to account for 60 to 90% of cognitive impairment diseases. This disease not only causes the memory loss of the patient but also destroys the personality and causes the patient to lose social function, so it is becoming a social problem.

In Japan, Donepezil hydrochloride, an anti-acetylcholinesterase inhibitor, was approved at the end of 1999, and it has become possible to “delay” the deterioration of cognitive function with a high probability if administered early. For Alzheimer's disease, early diagnosis is the most important issue in order to improve effects of current therapeutic methods and therapeutic drugs that will be developed in the future.

The American Psychiatric Association Key Diagnostic Criteria for Alzheimer's Disease (DSM IV) are listed below.
A. A multimodal manifestation of cognitive deficits manifested by both:
   (1) memory impairment (impaired ability to learn new information or recall previously learned information)
   (2) one or more of the following cognitive impairments
      a) aphasia (impairment of language)
      b) apraxia (impaired ability to perform movements in the absence of motor impairment)
      c) agnosia (impaired ability to recognize or identify objects in the absence of impaired sensory function)
      d) impaired ability to execute (plan, organize, sequence, abstract)
B. Cognitive deficits according to Criteria A (1) and A (2) each cause significant impairment in social or occupational functioning, showing marked decline from premorbid functional level (Edited by Imaharu Nakano and Hidehiro Mizusawa: Understanding Alzheimer's Disease, 2004, Nagai Shoten).

There are various diseases related to Alzheimer's disease (hereinafter also referred to as AD). In dementia such as AD, cognitive function gradually declines, so there is a state that should be called a prodromal state of dementia. Such a state is called mild cognitive impairment (hereinafter also referred to as MCI). Data from the United States indicate that 10 to 15% of MCI patients who visit a forgetfulness clinic will progress to AD within one year, and approximately 50% within four years. Most of the prodromal states of AD are involved in amnestic MCI.

According to the current definition, MCI is characterized by complaints of cognitive decline, but not interfering with basic activities of daily living. Frontotemporal dementia (FTD) is characterized by declining cognitive function and behavior that goes its own way without worrying about the surroundings, which is in contrast to AD where people try to fit in with their surroundings. FTD includes Pick's disease in which the presence of Pick's spheres is histologically confirmed in the cerebral cortex.

Dementia with Lewy bodies (DLB) is characterized by progressive memory impairment and visual cognitive impairment, including visual hallucinations. Diagnosed from clinical symptoms, DLB accounts for 10 to 30% of cases of dementia, and is the to be the second most common degenerative dementia disease in the elderly after Alzheimer-type dementia (AD). Histologically, it is characterized by the presence of Lewy bodies in the cerebrum. FTD and DLB are also called dementia-type neurological diseases because dementia is recognized and dementia-type (“Understanding Alzheimer's disease” described above).

Examinations widely used for diagnosing dementia are the revised Hasegawa Intelligence Rating Scale (HDS-R) and MMSE (Mini-Mental State Examination), which are based on interviews with subjects and determinations made based on the results. HDS was revised in 1991 to be called HDS-R.

It consists of 9 questions and tests orientation, memory, numeracy, memory/recall and common sense. A score of 23 or less out of a maximum of 30 points indicates suspected dementia. The MMSE was devised in the United States for the diagnosis of dementia, and includes orientation, memory, arithmetic, verbal, and graphic skills. It consists of 11 questions with a maximum score of 30 points, and dementia is suspected with a score of 23 or less, similar to the HDS-R. The results of both tests are said to be in good agreement in proportion. These interviews are used only for screening purposes and do not lead to a definitive diagnosis, and neither HDS-R nor MMSE is used for classification of a degree of severity (“Understanding Alzheimer's Disease” described above).

The diagnostic imaging methods include CT/MRI to see morphological abnormalities of the brain such as cerebral atrophy, sulcal ventricular enlargement and the like, cerebral perfusion scintigraphy (SPECT) to see cerebral blood flow, and positron emission tomography (PET) to see oxygen consumption and glucose consumption. SPECT and PET are nuclear medicine methods that are said to be able to detect morphological abnormalities before they occur (“Understanding Alzheimer's Disease” described above). However, since these image diagnoses require special equipment, they have the drawback that they cannot be performed at all medical institutions. In addition, the determination may differ depending on the doctor who sees the image, which lacks objectivity.

In this way, the diagnosis of dementia, including AD, currently relies on methods that lack objectivity and require the use of expensive equipment, making screening for disease detection impossible. If a biomarker could be found now that would allow objective diagnosis using easily obtainable patient samples such as blood (including serum and plasma), screening enables early detection of cognitive impairment diseases, which is currently the most important issue.

### 2. Combination of Biomarkers

The present technology provides a combination of biomarkers (a) to (e) described above. The present technology also provides a combination of the biomarkers (a) to (f) mentioned above. The amino acid sequences of these biomarkers are as follows. Combinations of biomarkers according to the present technology may be used for the detection, diagnosis, or determination of cognitive impairment or risk thereof.

The amino acid sequences of SEQ ID NOs: 1 to 6 described in (a) to (f) above are as described below.

### [1] Apolipoprotein A1-Derived Peptide (ApoA1) (SEQ ID NO: 1)

```
[intact protein/peptide]
0001 MKAAVLTLAV LFLTGSQARH FWQQDEPPQS
          PWDRVKDLAT VYVDVLKDSC
0051 RDYVSQFEGS ALGKQLNLKL LDNWDSVTST
          FSKLREQLGP VTQEFWDNLE
0101 KETEGLRQEM SKDLEEVKAK VQPYLDDFQK
          KWQEEMELYR QKVEPLRAEL
0151 QEGARQKLHE LQEKLSPLGE EMRDRARAHV
          DALRTHLAPY SDELRQRLAA
0201 RLEALKENGG ARLAEYHAKA TEHLSTLSEK
          AKPALEDLRQ GLLPVLESFK
0251 VSFLSALEEY TKKLNTQ
```

### [2] Transthyretin-Derived Peptide (TTR) (SEQ ID NO: 2)

| [intact protein/peptide] | |
|---|---|
| 0001 | MASHRLLLLC LAGLVFYSEA GPTGTGESKC |
| | PLMVKVLDAV RGSPAINVAY |
| 0051 | HVFRKAADDT WEPFASGKTS ESGELHGLTT |
| | EEEFVECIYK VEIDTKSYWK |
| 0101 | ALGISPFHEH AEVVFTANDS GPRRYTIAAL |
| | LSPYSYSTTA VVTNPKE |

### [3] Complement C3-Derived Peptide (C3) (SEQ ID NO: 3)

```
[intact protein/peptide]
0001 MGPTSCPSLL LLLLTHLPLA LGSPMYSIIT PNILRLESEE TMVLEAHDAQ
0051 CDVPYTVTVH DEPCKKLVLS SEKTVLTPAT NHMCNYTFTI PANREFKSEK
0101 CRNKFVIVQA TECTQVVEKV VLVSLQSGYL FIQTDKTIYT PCSTYLYRIF
0151 TVNHKLLPVG RTVMYNIENP EGIPVKQDSL SSQNQLGVLP LSWDIPELVN
0201 MGQWKIRAYY ENSPQQVFST EFEVKEYVLP SFEVIVEPTE KFYYTYNEKC
0251 LEVTITARFL YGKKVECTAF VIFGIQDCEQ RISLPESLKR IPIEDCSGEV
0301 VLSRKVLLDG VQNPRAEDLV GKSLYVSATV ILHSCSDMVQ AERSCIPIVT
0351 SPYQIHFTKT PKYFKPGMPF DLMVFVINPD GSPAYRVPVA VQGEDTVQSL
0401 TQGDGVAKLS INTHPSQKPL SITVRTKKQE LSEAEQATRT MQALPYSTVG
0451 NSNNYLHLSV LRTELRPCET LNVNFLLRMD RAHEAKIRYY TYLIMNKCRL
0501 LKAGRQVREP GQDLVVLPLS ITTDFIPSFR LVAYYTLIGA SGQREVVADS
0551 VWVDVKDSCV GSLVVKSGQS EDRQPVPCQQ MTLKIECDNG ARVYLVAVDK
0601 CVFVLNKKNK LTQSKIWDVV EKADIGCTPG SCKDYAGVFS DAGLTFTSSS
0651 GQQTAQRAEL QCPQPAARRR RSVQLTEKRM DKVGKYPKEL RKCCEDGMRE
0701 NPMRESCQRR TRFISLGEAC KKVFLDCCNY ITELRRQHAR ASHLGLARSN
0751 LDEDIIAEEN IVSRSEFPES WLWNVEDLKE PPKNGISTKL MNIFLKDSIT
0801 TWEILAVSNS DKKGICVADP FEVTYMQDFF IDLRLPYSVV RNEQVEIRAV
0851 LYNYRQNQEL KVRVELLHNP AFCSLATTKR RHQQTVTIPP KSSLSVPYVI
0901 VPLKTGLQEV EVKAAVYHHF ISDGVRKSLK VYPEGIRMNK TVAVRTLDPE
0951 RLGREGVQKE DIPPADLSDQ VPDTESETRI LLQGTPVAQM TEDAVDAERL
1001 KHLIVTPSGC GEQNMIGMTP TVIAVHYLDE TEQWEKFGLE KRQGALELIK
1051 KGYTQQLAFR QPSSAFAAFV KRAPSTWLTA YVVKVFSLAV NLIAIDSQVL
1101 CGAVKWLILE KQKPDGVFQE DAPVIHQEMI GGLENNNEKD MALTAFVLIS
1151 LQEAKDICEE QVNSLPGSIT KAGDFLEANY MNLQRSYTVA IAGYALAQMG
1201 RLKGPLLNKF LTTAKDKNRW EDPGKQLYNV EATSYALLAL LQLKDFDFVP
1251 PVVRWLNEQR YYGGGYGSTQ ATFMVFQALA QYQKDAPDHQ ELNLDVSLQL
1301 PSRSSKITHR IHWESASLLR SEETKENEGF TVTAECKGQG TLSVVTMYHA
1351 KAKDQLTCNK FDLKYTIKPA PETEKRPQDA KNTMILEICT RYRGDQDATM
1401 SILDISMMTG FAPDTDDLKQ LANGVDRYIS KYELDKAFSD RNTLIIYLDK
1451 VSHSEDDCLA FKYHQYFNVE LIQPGAVKVY AYYNLEESCT RFYHPEKEDG
1501 KLNKLCRDEL CRCAEENCFI QKSDDKVTLE ERLDKACEPG VQYVYKTRLV
1551 KVQLSNDFDE YIMAIEQTIK SGSDEVQVGQ QRTFISPIKC REALKLEEKK
1601 HYLMWGLSSD FWGEKPNLSY IIGKDTWVEH WPEEDECQDE ENQKQCQDLG
1651 AFTESMVVFG CPN
AB1-40:
SEQ ID NO: 4
DAEFRHDSGY EVHHQKLVFF AEDVGSNKGA IIGLMVGGVV
AB1-42:
SEQ ID NO: 5
DAEFRHDSGY EVHHQKLVFF AEDVGSNKGA IIGLMVGGVV IA
BACE1:
SEQ ID NO: 6
                10 20 30 40 50
MAQALPWLLL WMGAGVLPAH GTQHGIRLPL RSGLGGAPLG LRLPRETDEE
                60 70 80 90 100
PEEPGRRGSF VEMVDNLRGK SGQGYYVEMT VGSPPQTLNI LVDTGSSNFA
              110 120 130 140 150
VGAAPHPFLH RYYQRQLSST YRDLRKGVYV PYTQGKWEGE LGTDLVSIPH
              160 170 180 190 200
GPNVTVRANI AAITESDKFF INGSNWEGIL GLAYAETARP DDSLEPFFDS
              210 220 230 240 250
LVKQTHVPNL FSLQLCGAGF PLNQSEVLAS VGGSMIIGGI DHSLYTGSLW
              260 270 280 290 300
YTPIRREWYY EVIIVRVEIN GQDLKMDCKE YNYDKSIVDS GTTNLRLPKK
              310 320 330 340 350
VFEAAVKSIK AASSTEKFPD GEWLGEQLVC WQAGTIPWNI FPVISLYLMG
              360 370 380 390 400
EVINQSFRIT ILPQQYLRPV EDVATSQDDC YKFAISQSST GIVMGAVIME
              410 420 430 440 450
GFYVVFDRAR KRIGFAVSAC HVHDEFRIAA VEGPFVILDM EDCGYNIPQT
              460 470 480 490 500
DESTLMTIAY VMAAICALFM LPLCLMVCQW RCLRCLRQQH DDFADDISLL
K
```

### 3. Methods for Detecting, Diagnosing, or Determining Cognitive Impairment or Risk Thereof

The present technology also provides a method of detecting, diagnosing, or determining cognitive impairment or risk thereof. The method may include a step of detecting, diagnosing, or determining cognitive impairment or risk thereof (hereinafter also referred to as “determination step”) based on amounts of biomarkers that make up the combination in a human biological sample. In the determination step, a degree of cognitive impairment progression may be determined.

In the determination step, preferably, based on amounts of biomarkers (a), (b), and (c) and the ratio of amounts of biomarkers (d) and (e) (for example, Aβ40/Aβ42 or Aβ42/Aβ40, particularly Aβ40/Aβ42), cognitive impairment or risk thereof may be detected, diagnosed, or determined, or a degree of progression of cognitive impairment may be determined. More preferably, in the determination step, based on amounts of biomarkers (a), (b), and (c), the ratio of amounts of biomarkers (d) and (e) (e.g., Aβ40/Aβ42 or Aβ42/Aβ40, in particular Aβ40/Aβ42), and an amount of a biomarker (f), cognitive impairment or risk thereof may be detected, diagnosed, or determined, or a degree of progression of cognitive impairment may be determined.

In one embodiment, the determination step may include:
an index value calculating step of generating an index value for determination based on amounts of biomarkers (a), (b), (c), (d), and (e) (preferably, amounts of biomarkers (a), (b) and (c) and the ratio of amounts of biomarkers (d) and (e), and even more preferably, amounts of biomarkers (a), (b) and (c), the ratio of amounts of biomarkers (d) and (e), and an amount of a biomarker (f)), and
a supporting information generating step for generating supporting information used for determining the presence or absence of cognitive impairment or the risk of cognitive impairment based on the above-described index value for determination.

The method may include an outputting step of outputting information indicative of the determination result thus generated.

In the index value calculating step, by substituting amounts of biomarkers (a), (b), (c), (d), and (e) (preferably, amounts of biomarkers (a), (b) and (c) and the ratio of amounts of biomarkers (d) and (e), even more preferably, amounts of biomarkers (a), (b) and (c), the ratio of amounts of biomarkers (d) and (e), and an amount of a biomarker (f)) into a predetermined discriminant (for example, a regression model described later), the index value for determination may be calculated. For example, in the index value calculating step, the information processing device may calculate the index value for determination based on amounts of biomarkers (a), (b), (c), (d), and (e), more preferably based on amounts of biomarkers (a), (b) and (c) and the ratio of amounts of biomarkers (d) and (e), even more preferably based on amounts of biomarkers (a), (b) and (c), the ratio of amounts of biomarkers (d) and (e), and an amount of a biomarker (f).

The discriminant may be, for example, a discriminant created by multivariate analysis, in particular, may be a discriminant obtained by performing multivariate analysis with an amount of each biomarker constituting the combination of biomarkers described above (or amount and ratio) as an explanatory variable and with the presence or absence of cognitive impairment as an objective variable.

The index value for determination may be any value within a predetermined value range. The predetermined value range may be a value range indicating that the closer the index value is to the value of one end point of the value range, the higher the possibility that the human does not have cognitive impairment or cognitive decline, and the closer the index value is to the value of the other end point of the value range, the higher the possibility that the human has (more advanced) cognitive impairment. Humans are initially healthy (NDC), then progress to MCI among cognitive impairment, and then to AD with further progressed cognitive impairment. Therefore, which value within the predetermined value range the index value takes is useful for grasping whether the person has cognitive impairment and/or a degree of progression of cognitive impairment.

To generate the discriminant, a population of humans with known presence or absence of cognitive impairment (especially humans who have been determined to have AD, MCI, or NDC) may be used. The number of humans constituting the population may be, for example, 50 or more, 60 or more, or 70 or more. Although the upper limit of the number of humans constituting the population is not particularly limited, it may be, for example, 500 or less, 400 or less, 300 or less, or 200 or less. By performing multivariate analysis using the presence or absence of cognitive impairment or cognitive decline in each human constituting the population, and an amount of biomarkers contained in the biological samples obtained from each human, the discriminant for calculating the index value is obtained, and in particular, the coefficient of each term (and the value of the constant term) of the discriminant is obtained.

The multivariate analysis may preferably be logistic regression analysis (especially multinomial logistic regression analysis). Also, the multivariate analysis may be another linear regression analysis. Also, the multivariate analysis may be multiclass classification, for example, neural networks or support vector machines may be used.

The predetermined value range may be appropriately set by those skilled in the art. One endpoint of the predetermined value range may be −100, −50, −10, −5, −1, 0, 1, 5, 10, 50, or 100, for example. The other endpoint of the predetermined value range may be 100, 50, 10, 5, 1, 0, −1, −5, −10, −50, or −100. The predetermined value range may be a range defined by these endpoints, such as 0 to 1, 0 to 50, 0 to 100, −1 to 1, or −100 to 100, but the values of the endpoints of the value range may be values other than these.

In the supporting information generating step, supporting information for determining the presence or absence or risk of cognitive impairment is generated based on the index value. The index value is used to generate supporting information that contributes to accurately determining the presence or absence or risk of cognitive impairment.

The supporting information may include, for example, one or more of determination results regarding the presence or absence of cognitive impairment, determination results regarding risk of cognitive impairment, and data used for these determinations.

For example, the predetermined value range may be divided into several intervals, each interval being assigned the presence of cognitive impairment, and a degree of progression in having cognitive impairment. For example, when the predetermined value range is divided into three intervals, the three intervals may be, for example, the NDC interval, the MCI interval, and the AD interval. The NDC interval corresponds to an interval without cognitive impairment. The MCI and AD intervals correspond to intervals with cognitive impairment. The AD interval shows more advanced cognitive impairment than the MCI interval.

In the supporting information generating step, it may be specified which of the plurality of intervals the calculated index value corresponds to. Then, in the supporting information generating step, information indicating the determination results of the presence or absence or risk of cognitive impairment for the human from whom the biological sample is derived may be generated according to the identified interval.

In one embodiment, in the determination step of the present disclosure (especially in the supporting information generating step), it may be determined whether the human from whom the biological sample is derived is in a healthy stage, a mild cognitive impairment stage, or a dementia stage. The determination (the supporting information generation) may be performed by the information processing device.

The information may include, for example, information indicating a determination result that the human does not have cognitive impairment (or a determination result that there is a high or low possibility that the human does not have cognitive impairment), or information indicating a determination result that the human has cognitive impairment (or a determination result that there is a high or low possibility that the human has cognitive impairment). Furthermore, the information may include a determination result that the human is in a state of MCI (or a determination result that there is a high or low possibility that the human is in a state of MCI), or a determination result that the human is in a state of AD (or a determination result that there is a high or low possibility that the human is in a state of AD).

In the present specification, the term “amount” of a biomarker may be an absolute amount or a relative amount of the biomarker in a biological sample. The relative amount is, for example, concentration. For example, the biomarker concentration may be the mass of the biomarker relative to an amount (volume or mass) of the biological sample.

In the present specification, the term “biological sample” may be a biological sample derived from a human, such as whole blood, plasma, or serum, preferably plasma or serum, and particularly preferably plasma. Plasma is preferred, for example, from the standpoint of the stability of the biomarker amount during biological sample storage.

In the present technology, Aβ40/Aβ42 may be used as the ratio of amounts of biomarkers (d) and (e) as described above, alternatively, Aβ42/Aβ40 may be used.

The method may include a data acquisition step of acquiring data regarding amounts of biomarkers that make up the combination. The data acquisition step may include a step of measuring amounts of the biomarkers in the human biological sample, or may include a step of acquiring the previously measured biomarker amount data. An example of the measurement method for the former case will be described later. In the latter case, for example, the biomarker amount data stored in an information processing device or recording medium may be acquired.

In the data acquisition step, variation in amounts of biomarkers that make up the combination may be measured, or data relating to the variation may be acquired. The data on the variation can be used to diagnose cognitive impairment or risk thereof even more accurately.

The above-mentioned method can accurately detect, diagnose, or determine cognitive impairment or risk thereof. The method is, for example, highly accurate and specific in detecting, diagnosing or determining cognitive impairment or risk thereof. In particular, the method enables accurate detection, diagnosis or determination of both MCI and AD.

For example, the method may perform detection, diagnosis or determination of cognitive impairment or risk thereof with an AUC value of ROC for discrimination between MCI and NDC of 0.70 or more, preferably 0.75 or more, particularly preferably 0.80 or more, and an AUC value of ROC for discrimination between AD and NDC of 0.70 or more, preferably 0.75 or more, particularly preferably 0.80 or more.

Furthermore, the method is also highly useful in determining drug effects. That is, the method may include a drug effect determination step of determining an effect of a drug used to prevent, treat, or remedy cognitive impairment or risk thereof. The determination step may include a step of determining an effect of the drug based on changes in amounts (or ratio) of biomarkers that make up the combination before and after administration of the drug.

The method may include a step of comparing amounts (or ratio) of biomarkers constituting the combination of NDC-derived biological samples with amounts (or ratio) of biomarkers constituting the combination of subject-derived biological samples. This comparison step is useful in determining cognitive impairment or risk thereof.

According to the present technology, cognitive impairment of a subject can be determined. Furthermore, according to the present technology, it is possible to evaluate a subject's cognitive impairment at a mild stage, which is useful for preventive medicine. Furthermore, when psychotherapy or drug therapy is administered to patients suffering from cognitive impairment, if the progression of the disorder is suppressed, it will be reflected also in an amount of proteins/partial peptides in biological samples such as serum or plasma. By measuring this, therapeutic effects can be evaluated and determined, and drug discovery target biomolecules can be screened.

In the present specification, “peptide” of “partial peptide of intact protein” may include “polypeptide” and “oligopeptide”.

The term “oligopeptide” generally refers to a compound having bound amino acids with a molecular weight of 10,000 or less, or a compound having several (2 or more) to about 50 or less amino acid residues.

The term “polypeptide” refers to a compound of bound amino acids having a molecular weight of 10,000 or more, or a compound having about 50 or more amino acid residues.

In the present specification, the partial peptide of intact protein refers to a peptide having a partial amino acid sequence that is part of the amino acid sequence of the intact protein.

The partial peptide of intact protein refers to a case in which it is produced as a partial peptide in the expression synthesis process by transcription and translation and a case in which after being synthesized as an intact protein, it is digested and degraded in vivo to be generated as a digestive degradation product peptide. One of the reasons for this is that the protein synthesis and control mechanisms are deregulated when the body is in a state other than normal, such as a cognitive impairment disease.

With the present technology, it is possible to evaluate, discriminate, etc. whether a subject is in a normal state or suffer from a cognitive impairment disease, using the expression, synthesis, and/or digestive degradation of proteins in the body as an index, and also to evaluate, discriminate, etc. a degree of progression in a case of suffering from cognitive impairment.

“Detection of cognitive impairment” in the present technology may means detection of whether or not a subject suffers from cognitive impairment, and may also be evaluation, discrimination, diagnosis, examination, or the like. In addition, the detection of cognitive impairment diseases of the present technology may also include evaluation of risk of a subject suffering from more serious cognitive impairment.

In the present technology, the intact proteins that can be used as biomarkers for detecting cognitive impairment diseases include Apolipoprotein A1 comprising an amino acid sequence represented by SEQ ID NO: 1, Transthyretin comprising an amino acid sequence represented by SEQ ID NO: 2, and Complement C3 comprising an amino acid sequence represented by SEQ ID NO: 3.

Partial peptides of these intact proteins can also be used as biomarkers for detecting cognitive impairment diseases.

The term “partial peptide of intact protein” in the present technology is intended to include peptide fragments of 5 or more amino acid residues derived from the intact protein and peptides generated during the synthesis or degradation process thereof.

The intact protein partial peptide that can be used as biomarkers for detecting cognitive impairment include, for example, a polypeptide including an amino acid sequence represented by SEQ ID NO: 1 (preferably an Apolipoprotein A1-derived polypeptide), a polypeptide including an amino acid sequence represented by SEQ ID NO: 2 (preferably a Transthyretin-derived polypeptide), and a polypeptide including an amino acid sequence represented by SEQ ID NO: 3 (a Complement C3-derived polypeptide).

In the present technology, a protein or peptide including an amino acid sequence in which one or several amino acids are deleted, substituted, or added in each amino acid sequence of the above-described biomarkers (a) to (f) may be used as a biomarker.

Here, “one or several” refers to “1 to 3,” “1 or 2,” and “1.”

The partial peptides used as biomarkers in the present technology include proteins or peptides containing amino acid sequences represented by SEQ ID NOs: 1 to 3, and also peptide fragments having 5 or more amino acid residues derived from these.

The reason why “peptide fragments having 5 or more amino acid residues” includes “5 or more amino acid residues” in the present technology is according to the description of N. Benkirane et al., J. Biol. Chem. Vol. 268, 26279-26285, 1993. N. Benkirane et al. report that peptide CGGGERA, in which the amino acid residue sequence IRGERA at the C-terminus (130-135) of histone H3 was deleted by substituting K for R peptide and IR, and CGG was bound to GERA instead, was recognized by an antibody obtained using peptide IRGERA as an immunogen. This indicates that antigenic recognition is achieved by peptides consisting of 4 or more amino acid residues.

In the present technology, the number of residues was increased by 1 to 5 or more in order to have generality other than the C-terminus of histone H3, but it is important to target such low-molecular-weight peptides when using immunological techniques such as immunoblotting, ELISA, immunoMS, and the like to detect and sort them.

A sugar chain may be added to an intact protein, or a partial peptide thereof. Proteins and partial peptides to which these sugar chains are added can also be used as biomarkers for detecting cognitive impairment.

In addition, in the present technology, biomarkers may be quantified, or their presence or absence may be decided qualitatively. At this time, if the biomarker concentration is equal to or higher than a predetermined measured value or is equal to or higher than the standard value for the non-cognitive disease patient group, it can be detected, diagnosed, etc. as cognitive impairment. In addition, by the biomarker qualitative procedure, positive-negative detection, diagnosis, etc. can be performed, and for example, when reacting with a biomarker to show color development, etc., it is regarded as positive.

Two-dimensional electrophoresis or two-dimensional chromatography (2D-LC) can be used as a method for separating biomarkers in biological samples such as serum and the like in the present technology. Chromatography used for two-dimensional chromatography may be selected from known chromatographies such as ion exchange chromatography, reverse phase chromatography, gel filtration chromatography and the like.

In addition, as a method for separating biomarkers with the present technology, it is also possible to quantify them by an SRM/MRM method using LC-MS, which combines chromatography (LC) and triple quadrupole mass spectrometry. The LC used at this time may be one-dimensional LC.

Furthermore, if an immunoMS method (see, Japanese Patent Application Laid-Open No. 2004-333274) in which an antibody against a protein or peptide of interest is bound to beads (including magnetic beads), the protein or peptide to be measured is captured by this, and then eluted from the beads and measured by mass spectrometry, is used as the method for separating biomarkers in the present technology, the presence or absence or amount of a target protein, protein fragment, or peptide can be easily evaluated without using two-dimensional electrophoresis or chromatography.

The type and amount of one or more proteins in a biological sample can be measured simultaneously or separately by various methods. If the target protein (including protein fragments and partial peptides thereof) has been identified and an antibody (primary antibody) against it has been obtained, the following method can be used.

In the present technology, it is preferable to perform measurement by any one or more methods of Immunoblotting; Western blotting; enzyme or fluorescent or radioactive labeling; mass spectrometry; immunoMS; and surface plasmon resonance.

In addition, the biomarkers of the present technology can be measured simultaneously or separately even if they differ in type or amount.

In the present technology, a large number of these proteins, peptides or peptide fragments can be appropriately measured at once by using 2D-LC-MALDI-TOF-MS, SRM/MRM, and immunoMS methods, which combine two-dimensional chromatography and mass spectrometry.

Here, in the present technology, Enzyme Linked Immunosorbent Assay (ELISA), Chemiluminescent Immunoassay (CLIA), RADioimmunoassay (RIA), enzyme activity assay, etc. are referred to as “enzymatic or fluorescent or radioactive labeling method”. These methods using antibodies are referred to as “enzymatic or fluorescent or radioactive labeled antibody methods”.

### <Method for Measuring Biomarker Amount>

In the following, examples of methods for measuring an amount of biomarkers that make up the combination are described.

### (1) Immunoblotting Method

This is the simplest method. A test biological sample (for example, serum or plasma) diluted in several stages is prepared, and a fixed amount (approximately 1 microliter) thereof is dropped onto a suitable membrane such as a nitrocellulose membrane and air-dried. After treatment with a blocking solution containing a protein such as BSA and the like, the cells are washed, reacted with a primary antibody, washed, then, reacted with a labeled secondary antibody for detecting the primary antibody. After washing the membrane, the label is visualized and the concentration is measured.

### (2) Western Blot Method

After conducting one- or two-dimensional gel electrophoresis including isoelectric point or SDS-PAGE, the separated proteins are once transferred to a suitable membrane, such as a PVDF membrane, and the same immunoblotting method as described above is operated to measure an amount of the target protein using the primary antibody and the labeled secondary antibody.

### (3) ELISA Method

An antibody against a protein or its partial peptide is bound to a carrier such as a microtiter plate that has been specially chemically modified in advance, and after serially diluting the sample, an appropriate amount is added to the antibody-bound microtiter plate and incubated. It is then washed to remove uncaptured proteins and partial peptides. A secondary antibody conjugated with a fluorescent or chemiluminescent substance or enzyme is then added and incubated.

Detection is evaluated and determined by adding each substrate and then measuring visible light from a fluorescent or chemiluminescent substance or enzymatic reaction. Substances that can bind to proteins or partial peptides thereof may be used instead of antibodies. For example, aptamers and the like can be used.

The present technology preferably uses substances (e.g., antibodies, aptamers, etc.) against the biomarkers explained in (a) to (f) above.

Furthermore, methods (see, JP-A-2006-308533) are exemplified below, but are not limited thereto.

### (4) Method Using Microarray (Microchip)

A microarray is a general term for a device in which substances capable of binding to a substance to be measured are arrayed and immobilized on a carrier (substrate). In the case of the present technique, antibodies or aptamers against proteins or partial peptides may be aligned and immobilized before use.

Measurement is carried out by adding a biological sample to an immobilized antibody or the like, binding a protein or partial peptide to be measured on a microarray, and then adding a secondary antibody bound to a fluorescent or chemiluminescent substance or enzyme, before incubation. For detection, after adding each substrate, visible light from the fluorescence or chemiluminescent substance or enzymatic reaction may be measured.

### (5) Mass Spectrometry

In mass spectrometry, for example, an antibody against a specific protein or its partial peptide is bound to microbeads or substrates (protein chips) that have been specially chemically modified in advance. Microbeads may be magnetic beads. The material of the substrate does not matter.

The antibodies to be used are all of (1) an antibody that recognizes only the full length of a specific protein, (2) an antibody that recognizes only a partial peptide, (3) an antibody that recognizes both a specific protein and its partial peptide, or combinations of (1) and (2), (1) and (3), or (2) and (3) above may also be used.

After serially diluting the sample with a stock solution or a buffer solution, an appropriate amount of this is added to antibody-bound microbeads or substrates and incubated. It is then washed to remove uncaptured proteins and partial peptides. After that, the proteins and partial peptides captured on the microbeads or substrate are analyzed by mass spectrometry using MALDI-TOF-MS, SELDI-TOF-MS, etc., and the mass numbers of the peaks of the proteins, protein fragments and partial peptides and the peak intensities are measured. A certain amount of an appropriate internal standard substance is added to the original biological sample, the peak intensity is measured, and the ratio of the peak intensity to that of the target substance is calculated to know the concentration in the original biological sample. This method is called an immunoMS method.

Alternatively, after diluting the sample with a stock solution or a buffer or removing a portion of the protein, the protein can be separated by HPLC and quantified by mass spectrometry using the electrospray ionization (ESI) method. At that time, the concentration in the sample can be known by absolute quantification by the SRM/MRM method using an isotope-labeled internal standard peptide.

In addition to the above methods, proteins and partial peptides can also be analyzed by a method using two-dimensional electrophoresis, a method using surface plasmon resonance, or the like.

The present technology also includes a method of subjecting a biological sample collected from a subject to two-dimensional electrophoresis or a surface plasmon resonance method and detecting a cognitive impairment disease using the presence or absence or amount of the biomarker as an index.

### 4. Methods of Using Combination of Biomarkers

The present technology also provides a method of using a combination of biomarkers for the detection of cognitive impairment or risk thereof. The combinations may be used, for example, for diagnosis of cognitive impairment in humans. The combination may also be used for determining a degree of progression of cognitive impairment in humans.

Particularly preferably, in the use method, amounts of biomarkers (a), (b) and (c) and a ratio Aβ40/Aβ42 of amounts of biomarkers (d) and (e) are used. More preferably, in the method, amounts of biomarkers (a), (b) and (c), the ratio Aβ40/Aβ42 of amounts of biomarkers (d) and (e), and an amount of a biomarker (f) are used. In the method, human cognitive impairment or risk thereof may be detected, a diagnosis of cognitive impairment may be made, or a degree of progression of cognitive impairment may be determined based on these amounts and ratios.

### 5. Determination System for Cognitive Impairment

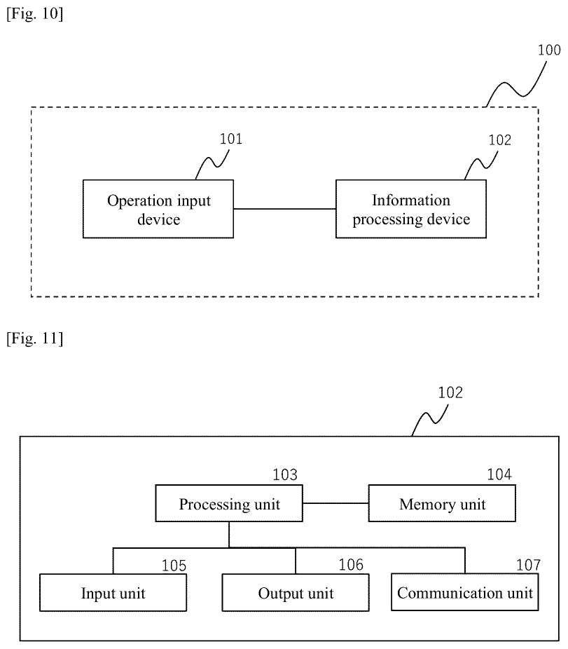

The present technology also provides a determination system for cognitive impairment. The determination system may be configured to perform the method explained in, for example, the above 3, or 4. A configuration example of the determination system is shown in FIG. 10. As shown in the figure, the determination system 100 according to the present technology may include, for example, a measurement system 101 that measures amounts of biomarkers that make up the combination, and an information processing device 102 for detecting, diagnosing, or determining cognitive impairment or risk thereof (or an information processing device 102 for determining a degree of progression of cognitive impairment) based on the biomarker amounts acquired by the measurement system. That is, the present technology also provides an information processing device that performs the method according to the present technology.

The measurement system may be configured to be able to perform the measurement explained in 3. described above, and in particular, includes a device configured to be able to perform any measurement method explained in <Method for measuring amount of biomarkers> in 3. above. The device desirably comprises, for example, an antibody- or aptamer-immobilizing unit (capturing portion) and a measurement unit. The antibody- or aptamer-immobilizing unit preferably has a solid-phase carrier such as a slide glass or a 96-well titer plate on which the antibody or aptamer is immobilized. In addition, it is preferable that the measurement unit is provided with a light detection means corresponding to a detection target, such as a spectrophotometer or a fluorescence spectrometer.

The information processing device 100 may include, for example, processing unit 103, storage unit 104, input unit 105, output unit 106, and communication unit 107, as shown in FIG. 11. The information processing device may be configured as, for example, a general-purpose computer or server, or may be configured as a cloud server.

For example, the processing unit may be configured to perform the determination step explained in 3. described above. In addition to the determination step, the processing unit may be configured to perform the data acquisition step explained in 3. described above.

The processing unit may include, for example, CPU (Central Processing Unit) and RAM. The CPU and RAM may be interconnected, for example via a bus. An input/output interface may be further connected to the bus. The input unit, the output unit, and the communication unit may be connected to the bus via the input/output interface.

Furthermore, the processing unit may be configured to acquire data from the storage unit or record data to the storage unit. The storage unit stores various data. The storage unit may be configured to be able to store, for example, the data acquired in the data acquisition step, the data related to the determination result in the determination step, and the like. In addition, the storage unit may store an operating system (for example, WINDOWS (registered trademark), UNIX (registered trademark), or LINUX (registered trademark)), a program for making an information processing device execute a method or information processing according to the present technology, and various other programs. Note that these programs may be recorded on a recording medium, not limited to the storage unit. That is, the present technology also provides a program for making an information processing device execute the determination step according to the present technology, and a recording medium storing the program.

The input unit may include an interface configured to receive input of various data. The input unit can include, for example, a mouse, a keyboard, a touch panel, etc. as a device that receives such operations.

The output unit may include an interface configured to output various data. For example, the output unit can output the determination result in the determination step. The output unit can include, for example, a display device and/or a printing device as a device that performs the output.

The communication unit may be configured to connect the information processing device to a network by wire or wirelessly. The communication unit enables the information processing device to acquire various data (for example, biomarker amount data and the like acquired in the data acquisition step) via a network. The acquired data can be stored, for example, in the storage unit. The configuration of the communication unit may be appropriately selected by those skilled in the art.

The information processing device may include, for example, a drive (not shown). The drive can read data (for example, the various data mentioned above) or programs recorded on the recording medium and output them to the RAM. The recording medium is, for example, a microSD memory card, an SD memory card, or a flash memory, but is not limited to these.

### 6. Biomarker Measurement Kit for Determining Cognitive Impairment

The present technology also provides a biomarker measurement kit constituting a combination of biomarkers according to the present technology. The measurement kit may contain, for example, an antibody or aptamer against the biomarker.

### 7. Regression Model Deciding Method

The present technology also provides a regression model deciding method for detecting cognitive impairment or risk thereof. The deciding method may include a step of acquiring or measuring data on amounts of biomarkers that constitute the combination contained in biological samples of each of a plurality of humans, and a regression analysis step of performing application into a regression model by conducting regression analysis, using the presence or absence of cognitive impairment or a stage of cognitive impairment in each of the plurality of humans and an amount measured for each human. The deciding method may include a detection step of detecting cognitive impairment or risk thereof in the subject using the regression model acquired by the application in the regression analysis step. Further, the deciding method may be executed, for example, by the information processing explained in 5. described above.

The regression model in the regression analysis step is, for example, logistic regression analysis, but is not limited thereto. In the regression model, the objective variable may be, for example, the presence or absence of cognitive impairment or a stage of cognitive impairment. In the regression model, the explanatory variable may be, for example, amounts of biomarkers that make up the combination according to the present technology or the ratio mentioned above.

### EXAMPLES

Hereinafter, the present technology will be explained in further detail based on examples. In addition, the examples described below show examples of typical examples of the present technology, and the scope of the present technology is not limited only to these examples.

### Test Example 1: Clinical Efficacy of Combination of Biomarkers

### Background

Blood biomarkers for MCI and preclinical stage offer important opportunities for the prevention of AD. Plasma AR correlates with brain amyloid accumulation, but its clinical utility in detecting early cognitive impairment is unclear.

### Method

Of a total of 681 specimens from a multicenter clinical study, 363 specimens (AD: 178, MCI: 145, cognitively healthy elderly (NDC): 40) diagnosed according to ADNI neuropsychological examination criteria were analyzed. The Aβ40 concentration, Aβ42 concentration, BACE1 concentration, and Triple marker (ApoA-1, C3, and TTR) concentrations in plasma samples were measured. The results of the measurements were used to determine the clinical effectiveness of these combinations of biomarkers in detecting cognitive impairment.

### Results and Discussion

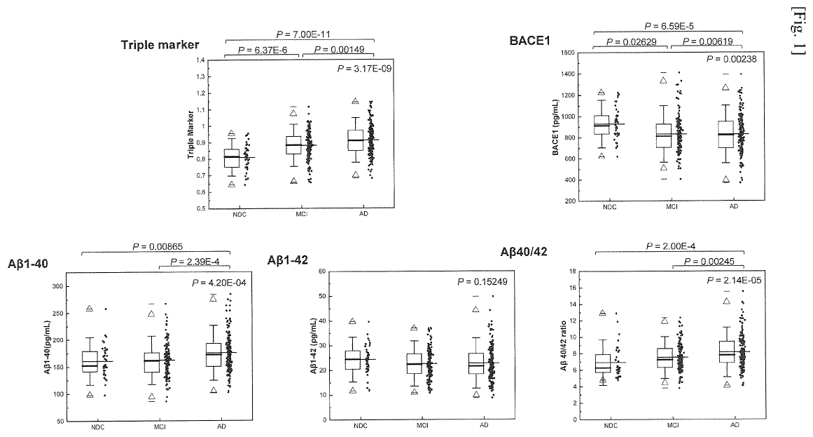

FIG. 1 shows the analysis results of the clinical effectiveness of the Triple marker score, BACE1 concentration, Aβ40 concentration, Aβ42 concentration, and Aβ40/42 ratio in detecting MCI or AD.

The Triple marker scores were acquired as follows. That is, regression analysis was performed using the Triple marker concentration of each of the 363 specimens and the information that each specimen was either MCI or NDC or information that each specimen was either AD or NDC. A discriminant was obtained by the analysis. The discriminant was set to have the value range shown in FIG. 1. By substituting the concentration of each specimen into these discriminants, the Triple marker score for each specimen was obtained.

As shown in FIG. 1, the Aβ40 concentration and Aβ40/42 ratio are excellent in detecting AD, but difficult to detect MCI. Triple markers are excellent for detecting NDC and MCI. Therefore, by combining these, it is possible to detect cognitive impairment widely from MCI to AD with high clinical efficacy.

That is, the combination of the three biomarkers ApoA-1, C3, and TTR, plus the biomarker Aβ40 or the biomarker ratio Aβ40/42 is useful to determine the presence or absence of cognitive impairment (particularly MCI and AD) or to determine risk of cognitive impairment in humans. Furthermore, the combination of the three biomarkers ApoA-1, C3, and TTR, plus the biomarker Aβ40 or the biomarker ratio Aβ40/42 is also useful for determining which stage a human is in between NDC to AD.

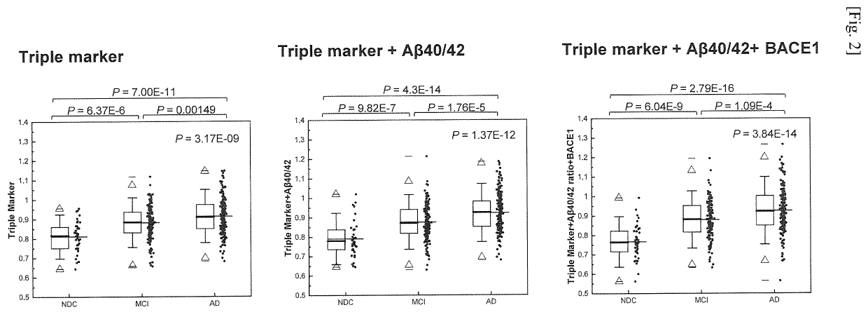

FIG. 2 shows the results of differential analysis by a Triple marker score, a score based on the Triple marker concentration and the Aβ40/42 ratio (also referred to as Triple marker+Aβ40/42 score), and a score based on the Triple marker concentration and the Aβ40/42 ratio and the BACE1 concentration (also referred to as Triple marker+Aβ40/42+BACE1 score).

The Triple marker score is as described with respect to FIG. 1 above.

The Triple marker+Aβ40/42 score was acquired as follows. That is, using the Triple marker concentration and Aβ40/42 ratio of each of the 363 specimens, and information on whether each specimen is MCI or NDC or information on whether each specimen is AD or NDC, regression analysis was performed to obtain the discriminant. The discriminant was set to have the value range shown in FIG. 2. By substituting the concentration of each specimen into the discriminant, the Triple marker+Aβ40/42 score of each specimen was obtained.

The Triple marker+Aβ40/42+BACE1 score was acquired as follows. That is, using the Triple marker concentration, Aβ40/42 ratio, and BACE1 concentration of each of the 363 specimens, information on whether each specimen is MCI or NDC, or information on whether each specimen is AD or NDC, logistic regression analysis was performed to obtain the discriminant. The discriminant was set to have the value range shown in FIG. 2. By substituting the concentration of each specimen into the discriminant, the Triple marker+Aβ40/42+BACE1 score of each specimen was obtained.

As shown in FIG. 2, the Triple marker+Aβ40/42 score is better than the Triple marker score for differentiation between NDC, MCI and AD. In addition, the Triple marker+Aβ40/42+BACE1 score is also superior to the Triple marker score and the Triple marker+Aβ40/42 score for differentiation between NDC, MCI, and AD, and P-value indicating a significant difference between the three groups is increased by 5 orders of magnitude.

These results indicate that the combination of Triple marker and Aβ40/42 ratio is suitable for differentiation between NDC, MCI and AD. Also, it can be seen that the combination of Triple marker, Aβ40/42 ratio and BACE1 is even more suitable for differentiation between NDC, MCI and AD.

That is, the combination of the Triple marker and the Aβ40/42 ratio is useful for determining the presence or absence of cognitive impairment (especially MCI and AD) or for determining risk of cognitive impairment in humans. The combination of Triple marker, Aβ40/42 ratio and BACE1 is further useful for determining the presence or absence of cognitive impairment (particularly MCI and AD) or for determining risk of cognitive impairment in humans.

As described above, according to the combination of biomarkers according to the present technology, detection of cognitive impairment or detection of risk of cognitive impairment can be performed with higher accuracy.

### Test Example 2: Evaluation by ROC

To confirm the efficacy in diagnosing cognitive impairment of the combination of Triple marker concentration and Aβ40/42 ratio and the combination of Triple marker concentration, Aβ40/42 ratio and BACE1 concentration, evaluation was performed by ROC (Receiver Operating Characteristic) analysis. In addition, evaluation by ROC analysis was also performed in a case of using only the concentration of the Triple marker and the case of using only the ratio of Aβ40/42 in the evaluation. These analyses were performed on the specimens used in Test Example 1 above.

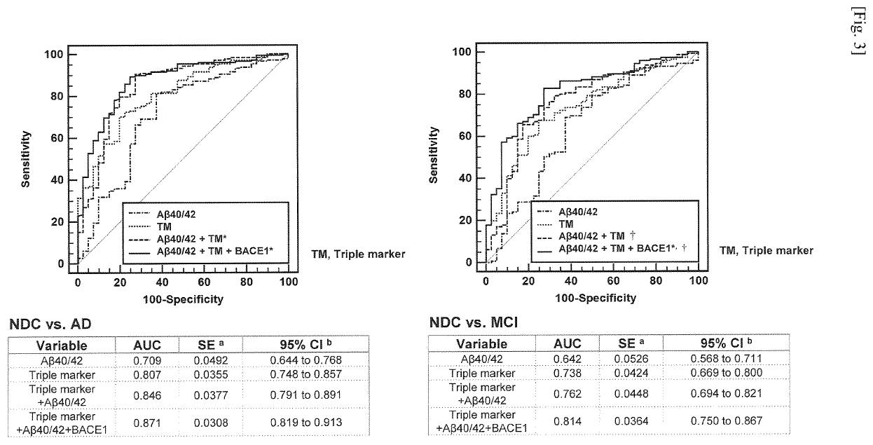

ROC curves regarding NDC and MCI discrimination and NDC and AD discrimination by the Aβ40/42 ratio, Triple marker score, Triple marker+Aβ40/42 score, or Triple marker+Aβ40/42+BACE1 score are shown in FIG. 3. Also shown in FIG. 3 are the AUC (area under the curve) of the ROC curve, SE (standard error), and 95% CI (confidence interval).

In FIG. 3, * indicates significantly higher clinical efficacy compared to Aβ40/42 ratio and Triple marker score. † indicates significantly higher clinical efficacy compared to Aβ40/42.

As shown in FIG. 3, the AUC value increases in the order of the Aβ40/42 ratio, Triple marker score, Triple marker+Aβ40/42 score, and Triple marker+Aβ40/42+BACE1 score, both in NDC and MCI discrimination and in NDC and AD discrimination. That is, Triple marker+Aβ40/42 score is more effective in diagnosing MCI and AD than Aβ40/42 ratio and Triple marker score. Triple marker+Aβ40/42+BACE1 score is more effective in diagnosing MCI and AD than Aβ40/42 ratio and triple marker score, and more effective in diagnosing than Triple marker+Aβ40/42 score.

Therefore, the combination of the three biomarkers ApoA1, C3, and TTR with the biomarker ratio Aβ40/42 is useful to determine the presence or absence of cognitive impairment (particularly MCI and AD) or to determine risk of cognitive impairment in humans. Furthermore, the combination of the three biomarkers ApoA-1, C3, and TTR with the biomarker ratios Aβ40/42 and BACE1 is further effective for determining the presence or absence of cognitive impairment (especially MCI and AD) or for determining risk of cognitive impairment in humans.

Details of the regression analysis used in the above test examples are described below. Although logistic regression analysis is described below, the discriminant for calculating the score used for discrimination may be derived by analysis other than logistic regression analysis.

### <Distinction Between MCI and AD by Multimarkers Using Logistic Regression Analysis>

### (1) Principle of Logistic Regression Analysis

This method obtains the coefficients of each parameter corresponding to the biomarkers from the dataset and can give a determination probability for each patient in two disease categories (normal, disease). A relatively thorough discussion of logistic regression can be found here (Czepiel, SA, http://czep.net/stat/mlelr.pdf, 2010, Maximum likelihood estimation of logistic regression models: theory and implementation). This commentary includes analytical methods by the Newton-Raphson method.

The principle of this analysis method is as follows. It is known that when a probability of occurrence of a certain event is represented by P, then

$$\begin{matrix}[ Number 1 ] & \\ F ( Z ) = p = \frac{1}{1 + {e}^{- z}} & ( 1 )\end{matrix}$$

can be approximated by the cumulative standard normal distribution (Bowling, S R, et al. JIEM, 2009, 2: 114-127, A logistic approximation to the cumulative normal distribution). Logistic regression uses this approximation to perform statistical analysis. Z is expressed as a linear combination of multivariate xᵢ; i=1, 2, . . . r as follows.

$$\begin{matrix}[ Number 2 ] & \\ Z = {β}_{0} + {β}_{1} {x}_{1} + {β}_{2} {x}_{2} + … + {β}_{r} {x}_{r} & ( 2 )\end{matrix}$$

A large number of data are applied into to equations (1) and (2) to obtain coefficient βᵢ, and its significance (βᵢ is not zero) is determined from statistical p-values.

There are two ways to finalize the formula (2).

### (I) Method by Statistical Significance of Coefficients

If a non-significant coefficient is found, it is discarded to form the equation (2) and the application is repeated in the same manner. Thus, when all the coefficients of the formula (2) become significant, the Z value is obtained from (2) by substituting the measured values of xi. The value of P (determination probability) can then be obtained from the equation (1). Note that the standard error of the coefficient βᵢ can be calculated.

(II) Method for Obtaining the Combination of Coefficients that Give the Maximum Ratio of Correct Answers

The ratio of correct answers refers to the rate of correctly determined to belong to the original group, but in this method, the combination of coefficients that gives the maximum ratio of correct answers by trial and error for all coefficients, including coefficients with no statistical significance, is obtained. The determination probability is obtained in the same way as in (I).

The ratio of correct answers in logistic regression is defined by the following equation (3). Discrimination is performed by estimating to which of two categories (e.g., NDC and MCI) a subject belongs from a logistic regression equation. When the subject's category is set to i (e.g., MCI) and when a determination probability obtained from the logistic regression equation is 0.5 or more, it is considered that i is correctly diagnosed. When the total number of subjects in the category i is represented by Ni and the number of subjects correctly diagnosed as i is represented by Cᵢ, then, the ratio of correct answers is represented as follows.

$$\begin{matrix}[ Number 3 ] & \\ Ratio of correct answers = {C}_{i} / {N}_{i} & ( 3 )\end{matrix}$$

In logistic regression, the odds ratio for a variable xᵢ is the odds of increasing xᵢ by one unit divided by the original odds, which is equal to exp (βᵢ). An odds ratio of 1 means that even if xᵢ is increased by one unit, the odds are the same as the original, so there is no change in a probability of the event under consideration. That is, βᵢ is 0 in this case, indicating that it does not contribute to Z. Even if the 95% confidence interval of the odds ratio includes 1, the βᵢ is not statistically significant. Note that exp (0)=1, of course.

### (2) Logistic Regression Analysis

In the above test examples, logistic regression analysis was performed on the biomarkers measured by the multiplex immunoassay method. In the analysis by logistic regression analysis, marker proteins are added or subtracted by trial and error, the combination of marker proteins that yields the highest ratio of correct answers is found, and the coefficient (βᵢ) of the logistic regression equation (2) by this combination was calculated.

Logistic regression analysis was performed using MedCalc for Windows, version 9, 2007, (MedCalc Software). This program relies on the Newton-Raphson method.

Moreover, the detailed data regarding Test examples 1 and 2 above are described below.

Table 1 below shows the results of biomarker analysis of plasma samples in 363 specimens from the multicenter clinical study. The table shows the average values for each of NDC, MCI, and AD.

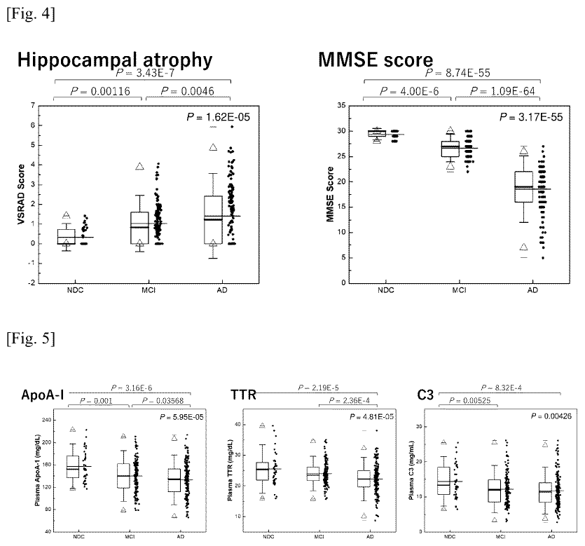

Further, the distributions of VSRAD and MMSE scores for NDC, MCI, and AD, respectively, are shown in FIG. 4, and plasma concentrations of ApoA-1, TTR, and C3 are shown in FIG. 5.

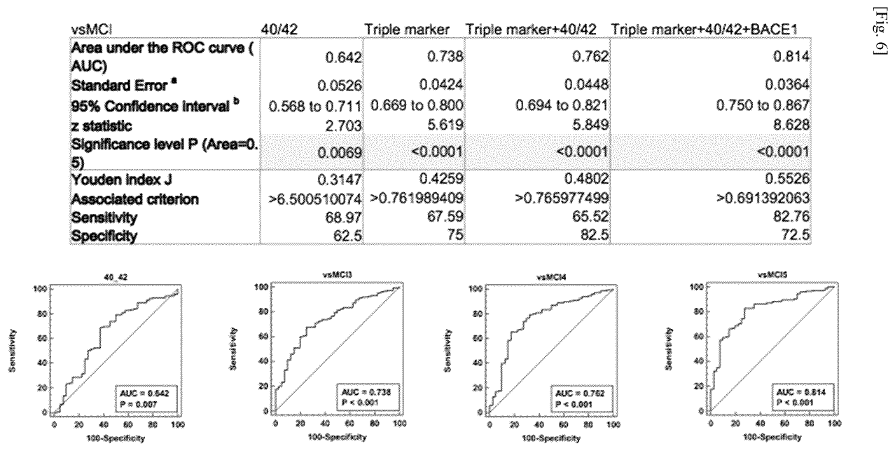

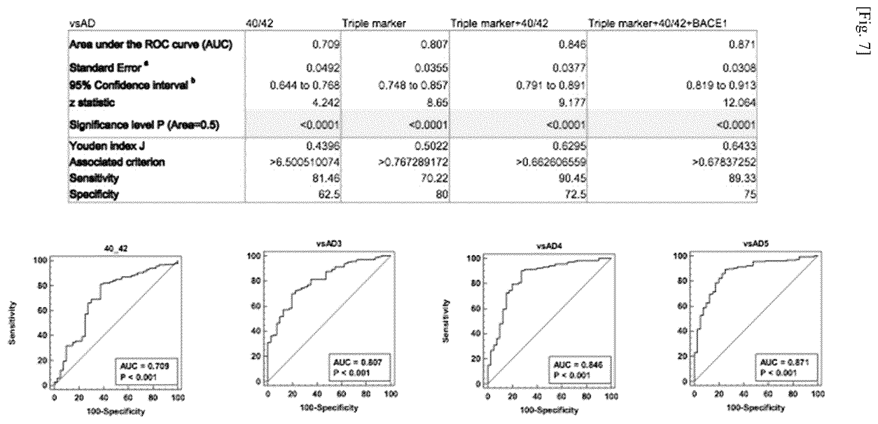

Further, FIG. 6 shows details of the results of ROC analysis for the discrimination results of NDC and MCI, and FIG. 7 shows details of the results of ROC analysis of the discrimination results of NDC and AD.

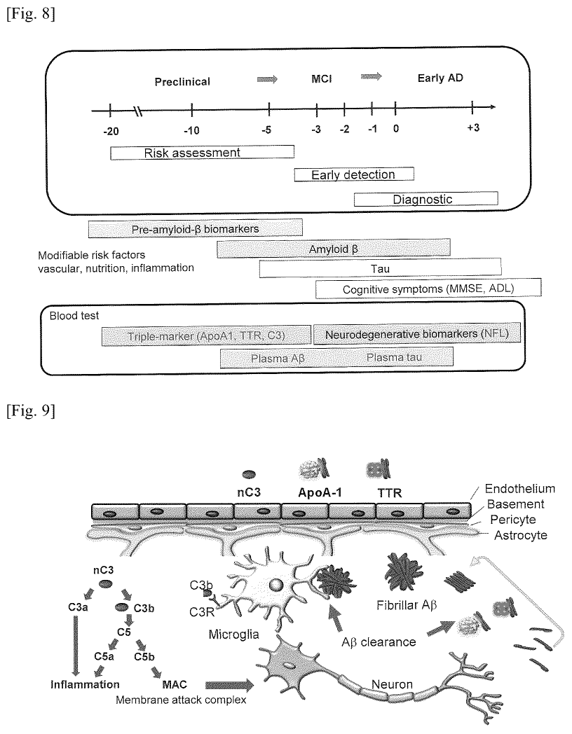

FIG. 8 shows the Context of Use of biomarkers in the pathophysiology of AD, and FIG. 9 shows the action of Sequester protein as biomarkers (especially blood biomarker) associated with cognitive decline.

TABLE 1
Results of biomarker analysis of plasma samples
in 363 specimens from multicenter clinical study

| | NDC | MCI | AD | P value* |
|---|---|---|---|---|
| Number of subjects | 40 | 145 | 178 | |
| Memory Clinic | 33 | 89 | 74 | |
| Uji hospital | 7 | 17 | 3 | |
| Fukushimura hospital | — | 22 | 75 | |
| Nagoya city University | — | 9 | 21 | |
| Tsukuba University | — | 8 | 5 | |
| Age** | 66.4 ± 8.6 | 73.9 ± 7.7 | 79.4 ± 7.3 | 5.94E−18 |
| Gender (M/F) | 7/33 | 77/68 | 55/123 | |
| ApoA-1** | 157.9 ± 26.6 | 140.3 ± 30.1 | 133.3 ± 29.7 | 5.94E−05 |
| TTR** | 25.5 ± 5.3 | 24.0 ± 3.8 | 22.2 ± 4.7 | 4.81E−05 |
| Plasma C3** | 14.4 ± 4.7 | 12.2 ± 4.5 | 11.8 ± 4.5 | 0.00426 |
| Aβ1-40** | 160.7 ± 29.3 | 162.2 ± 29.8 | 175.5 ± 34.1 | 4.20E−04 |
| Aβ1-42** | 24.4 ± 6.0 | 22.6 ± 6.0 | 22.7 ± 6.8 | 0.15249 |
| Aβ40/42 ratio** | 6.9 ± 1.8 | 7.5 ± 1.7 | 8.1 ± 2.0 | 2.14E−05 |
| BACE1** | 930.2 ± 150.1 | 834.4 ± 178.5 | 832.8 ± 182.1 | 0.00238 |
| Triple-marker** | 0.81 ± 0.07 | 0.88 ± 0.08 | 0.91 ± 0.09 | 3.17E−09 |
| Triple-marker + Aβ40/42 ratio** | 0.79 ± 0.08 | 0.88 ± 0.09 | 0.92 ± 0.10 | 1.37E−12 |
| Triple-marker + Aβ40/42 + BACE1** | 0.77 ± 0.09 | 0.88 ± 0.10 | 0.93 ± 0.12 | 3.48E−14 |
| VSRAD*** | 0.33 ± 0.46 | 1.03 ± 0.95 | 1.41 ± 1.43 | 1.62E−05 |
| MMSE** | 29.4 ± 0.7 | 26.7 ± 1.8 | 18.6 ± 4.4 | 3.17E−55 |

*Significant differences among 3 groups (Kruskal-Wallis test)
**mean ± SD
***VSRAD, Voxel-based Specific Regional analysis system for Alzheimer Disease

1. A system for determining cognitive impairment, comprising an information processing device that executes a determination step of determining the presence or absence or risk of cognitive impairment based on amounts of the following biomarkers (a), (b), (c), (d), and (e) contained in a biological sample:
(a) a biomarker consisting of an intact protein of Apolipoprotein A1 comprising an amino acid sequence represented by SEQ ID NO: 1, or a partial peptide thereof;
(b) a biomarker consisting of an intact protein of Transthyretin comprising an amino acid sequence represented by SEQ ID NO: 2, or a partial peptide thereof;
(c) a biomarker consisting of an intact protein of Complement C3 having an amino acid sequence represented by SEQ ID NO: 3, or a partial peptide thereof;
(d) a biomarker Aβ1-40 consisting of a peptide having an amino acid sequence represented by SEQ ID NO: 4, and
(e) a biomarker Aβ1-42 consisting of a peptide having an amino acid sequence represented by SEQ ID NO: 5.

2. The determination system according to claim 1, wherein the information processing device determines presence or absence of cognitive impairment or risk of cognitive impairment based on amounts of biomarkers (a), (b), and (c) and a ratio of amounts of biomarkers (d) and (e).

3. The determination system according to claim 2, wherein the information processing device is configured to determine presence or absence of cognitive impairment or risk of cognitive impairment, further based on an amount of the following biomarker (f), in addition to the amounts of biomarkers (a), (b), and (c) and the ratio of amounts of biomarkers (d) and (e):
(f) a biomarker consisting of an intact protein of BACE1 comprising an amino acid sequence represented by SEQ ID NO: 6, or a partial peptide thereof.

4. The determination system according to any one of claims 1 to 3, wherein the information processing device determines, in the determination step, whether a human from which the biological sample is derived is in a healthy stage, a mild cognitive impairment stage, or a dementia stage.

5. An information processing device that executes a determination step of determining the presence or absence or risk of cognitive impairment based on amounts of the following biomarkers (a), (b), (c), (d), and (e) contained in a biological sample:
(a) a biomarker consisting of an intact protein of Apolipoprotein A1 comprising an amino acid sequence represented by SEQ ID NO: 1, or a partial peptide thereof;
(b) a biomarker consisting of an intact protein of Transthyretin comprising an amino acid sequence represented by SEQ ID NO: 2, or a partial peptide thereof;
(c) a biomarker consisting of an intact protein of Complement C3 having an amino acid sequence represented by SEQ ID NO: 3, or a partial peptide thereof;
(d) a biomarker Aβ1-40 consisting of a peptide having an amino acid sequence represented by SEQ ID NO: 4, and
(e) a biomarker Aβ1-42 consisting of a peptide having an amino acid sequence represented by SEQ ID NO: 5.

6. A method for determining cognitive impairment, comprising a determination step of determining presence or absence of cognitive impairment or risk of cognitive impairment, based on amounts of the following biomarkers (a), (b), (c), (d), and (e) contained in a biological sample:
(a) a biomarker consisting of an intact protein of Apolipoprotein A1 comprising an amino acid sequence represented by SEQ ID NO: 1, or a partial peptide thereof;
(b) a biomarker consisting of an intact protein of Transthyretin comprising an amino acid sequence represented by SEQ ID NO: 2, or a partial peptide thereof;
(c) a biomarker consisting of an intact protein of Complement C3 having an amino acid sequence represented by SEQ ID NO: 3, or a partial peptide thereof;
(d) a biomarker Aβ1-40 consisting of a peptide having an amino acid sequence represented by SEQ ID NO: 4, and
(e) a biomarker Aβ1-42 consisting of a peptide having an amino acid sequence represented by SEQ ID NO: 5.

7. A biomarker combination comprising the following biomarkers (a), (b), (c), (d), and (e), for use in determining presence or absence of cognitive impairment or risk of cognitive impairment:
(a) a biomarker consisting of an intact protein of Apolipoprotein A1 comprising an amino acid sequence represented by SEQ ID NO: 1, or a partial peptide thereof;
(b) a biomarker consisting of an intact protein of Transthyretin comprising an amino acid sequence represented by SEQ ID NO: 2, or a partial peptide thereof;
(c) a biomarker consisting of an intact protein of Complement C3 having an amino acid sequence represented by SEQ ID NO: 3, or a partial peptide thereof;
(d) a biomarker Aβ1-40 consisting of a peptide having an amino acid sequence represented by SEQ ID NO: 4, and
(e) a biomarker Aβ1-42 consisting of a peptide having an amino acid sequence represented by SEQ ID NO: 5.


SEQUENCE LISTING (6 sequences)

SEQ ID NO: 1 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 2 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 3 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 4 - protein, 40 aa, Homo sapiens
DAEFRHDSGYEVHHQKLVFFAEDVGSNKGAIIGLMVGGVV

SEQ ID NO: 5 - protein, 42 aa, Homo sapiens [sequence omitted]

SEQ ID NO: 6 - protein, 501 aa, Homo sapiens
MAQALPWLLLWMGAGVLPAHGTQHGIRLPLRSGLGGAPLGLRLPRETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESTLMTIAYVMAAICALFMLPLCLMVCQWRCLRCLRQQHDDFADDISLLK